(19) United States
(12) Patent Application Publication
(10) Pub. No.: US 2022/0064707 A1
(43) Pub. Date: Mar. 3, 2022

(54) RAPID REVERSE TRANSCRIPTION QUANTITATIVE POLYMERASE CHAIN REACTION

(71) Applicant: Northwestern University, Evanston, IL (US)

(72) Inventors: Jennifer L. Reed, Chicago, IL (US); Sally M. McFall, Evanston, IL (US); Matthew A. Butzler, Arlington Heights, IL (US)

(21) Appl. No.: 17/421,677

(22) PCT Filed: Jan. 16, 2020
(86) PCT No.: PCT/US2020/013827

§ 371 Date: Jul. 8, 2021

Related U.S. Application Data
(60) Provisional application No. 62/793,701, filed on Jan. 17, 2019.

(51) Int. Cl. C12Q 1/686; C12Q 1/6806

(52) U.S. Cl. CPC C12Q 1/686; C12Q 1/6806

(57) ABSTRACT

Provided herein are methods for rapid detection of RNA in a sample. The methods comprise providing a reaction mixture containing the sample, amplification reagents, and a polymerase enzyme having both RNA and DNA-dependent polymerase activity; reverse transcribing the RNA to DNA by incubating for a reverse transcription time of no longer than 5 minutes; and amplifying the DNA by performing a thermal cycling protocol comprising a plurality of amplification cycles, wherein each amplification cycle comprises at least a denaturation step and an annealing step.

### CROSS-REFERENCE TO RELATED APPLICATION

The present application claims priority to U.S. Provisional Patent Application Serial No. 62/793,657, filed Jan. 17, 2019, which is hereby incorporated by reference in its entirety.

### FIELD

Provided herein are methods for rapid amplification and detection of RNA in a sample. In particular embodiments, The disclosed methods may be used for clinical diagnostics, such as for the detection of a viral infection in a subject.

### BACKGROUND

Infectious diseases are frequently diagnosed with nucleic acid tests. However, the most widely practiced methods for amplification of nucleic acids, polymerase chain reaction (PCR) or Quantitative or real-time PCR (qPCR or RT-PCR), are both time and energy intensive. The detection of RNA can also be essential for clinical diagnostics especially for viral infections, but RNA cannot be directly amplified by PCR. It requires a first step of reverse transcription where an enzyme called reverse transcriptase enzymatically makes a DNA copy (cDNA) from the RNA template. In RT-qPCR, this cDNA is then amplified in the PCR reaction.

RT-qPCR assays can be performed in either a one-step or two-step reaction. In one-step RT-qPCR, cDNA synthesis and qPCR are performed in a single reaction vessel in a common reaction buffer. In two-step RT-qPCR, cDNA is synthesized in one reaction, and an aliquot of the cDNA is then used for a subsequent qPCR experiment. One-step reactions allow for minimal sample handling and closed-tube reactions, reducing chances for pipetting errors and cross-contamination. However, for a single tube RT-qPCR assay, the combined RT and PCR reagents must allow these reactions to proceed together in one tube. This prevents use of the most optimal reagents and conditions for each individual reaction, thus potentially compromising reaction conditions and negatively affecting efficiency and yield.

Accordingly, improved methods for rapid detection of target RNA in a sample that allow for high efficiency and yield are needed.

### SUMMARY

Provided herein are methods for detecting a target RNA in a sample. In some embodiments, provided herein are methods for detecting a target RNA in a sample comprising: (a) providing a reaction mixture containing the sample, amplification reagents, and a polymerase enzyme having both RNA and DNA-dependent polymerase activity; (b) reverse transcribing the RNA to DNA by incubating for a reverse transcription time of no longer than 5 minutes; and (c) amplifying the DNA by performing a thermal cycling protocol comprising a plurality of amplification cycles, wherein each amplification cycle comprises at least a denaturation step and an annealing step.

In some embodiments, the amplification reagents comprise deoxynucleotide triphosphates, a buffer, a cofactor, and oligonucleotide primers configured for amplification of the target RNA in the sample. In some embodiments, the oligonucleotide primers comprise a forward primer and a reverse primer. In some embodiments, the oligonucleotide primers are provided at a concentration of at least 6 μM (e.g., 6 μM, 7 μM, 8 μM, 904, 10 μM, 11 μM, 12 μM, 13 μM, 14 μM, 15 μM, 16 μM, 17 μM, 18 μM, or ranges therebetween).

In some embodiments, the oligonucleotide primers are provided at a concentration of 12 μM. In some embodiments, the polymerase enzyme is provided at a concentration of at least 0.4 U/μL, (e.g., 0.4 U/μL, 0.5 U/μL, 0.6 U/μL, 0.7 U/μL, 0.8 U/μL, 0.9 U/μL, 1.0 U/μL, 1.1 U/μL, 1.2 U/μL or ranges therebetween). In some embodiments, the polymerase enzyme is provided at a concentration of 0.8 U/μL.

In some embodiments, the cofactor is a magnesium salt or a manganese salt. In some embodiments, the cofactor is a manganese salt. In some embodiments, the manganese salt is MnCl₂ In some embodiments, the cofactor is provided at a concentration of 3 mM to 8 mM (e.g., 3 mM, 4 mM, 5 mM, 6 mM, 7 mM, 8 mM, or ranges therebetween). In some embodiments, the cofactor is provided at a concentration of 4 mM.

In some embodiments, the reverse transcription time is no longer than 2 minutes. In some embodiments, the reverse transcription time is no longer than 30 seconds. In some embodiments, the reverse transcription time is no longer than 12 seconds. In some embodiments, the reverse transcription time is no longer than 5 seconds. In some embodiments, wherein the reverse transcription time is no longer than 1 second.

In some embodiments, the reverse transcribing step occurs at a temperature of 64-72° C. (e.g., 64° C., 65° C., 66° C., 67° C., 68° C., 69° C., 70° C., 71° C., 72° C., or ranges therebetween). In some embodiments, the reverse transcribing step occurs at a temperature of 68° C. . In some embodiments, each denaturation step is performed for 1 second at 91-99° C. (e.g., 91° C., 92° C., 93° C., 94° C., 95° C., 96° C., 97° C., 98° C., 99° C., or ranges therebetween). In some embodiments, each annealing step is performed for 4 seconds at 64-72° C. (e.g., 64° C., 65° C., 66° C., 67° C., 68° C., 69° C., 70° C., 71° C., 72° C., or ranges therebetween). In some embodiments, each denaturation step is performed for 1 second at 95° C. and each annealing step is performed for 4 seconds at 68° C. In some embodiments, the thermal cycling protocol comprises at least 30 amplification cycles (e.g., 30, 35, 40, 45, 50, 55, 60, or more, or ranges therebetween). In some embodiments, the thermal cycling protocol comprises 40 amplification cycles.

### BRIEF DESCRIPTION OF THE DRAWINGS

FIG. 1. Box and whisker plot of Cq of MS2 RT-qPCR with varying RT times in seconds. The number of replicates is indicated on graph.

FIG. 2. Amplification curves of MS2 RNA amplified with either Mg²⁺ or Mn²⁺ cofactors.

FIG. 3. Amplification curves of a plasmid containing the closed HCV cDNA and in vitro transcribed RNA from the plasmid with either Mg²⁺ or Mn²⁺ cofactors.

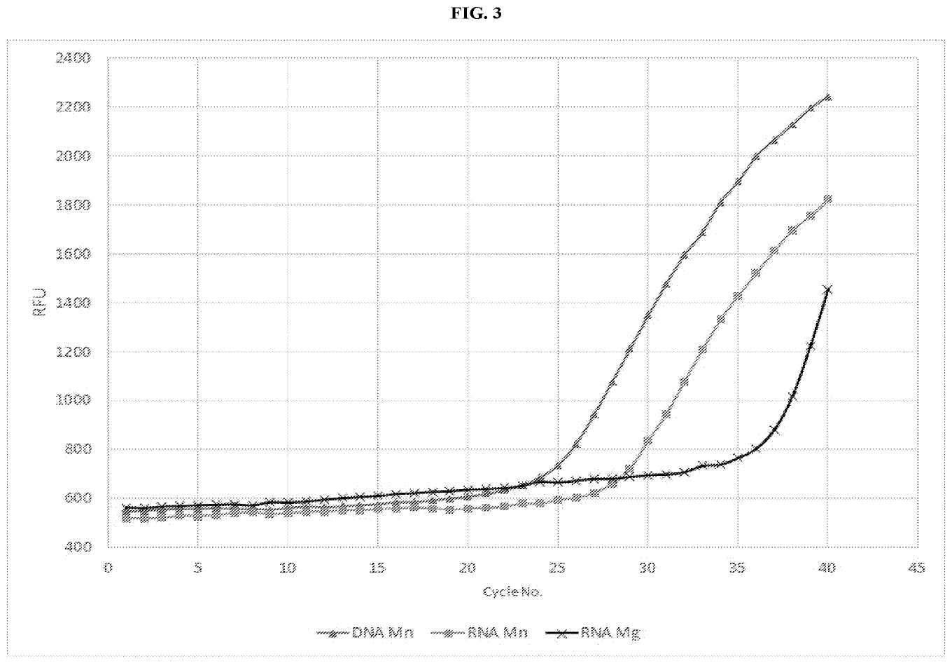

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “analyzing” and linguistic equivalents thereof refers to any steps taken to a characterize a sample or one or more components thereof. Exemplary analysis steps include, for example, quantification of a sample component (e.g., a target nucleic acid), sequencing a sample component, etc.

As used herein, the term “preparing” and linguistic equivalents thereof refers to any steps taken to alter a sample or one or more components thereof, for example, for use in a subsequence analysis or detection step. Exemplary sample preparation steps include, for example, dilution or concentration of a sample, isolation or purification of a sample component, heating or cooling a sample, amplification of a sample component (e.g., nucleic acid), labeling sample components, etc.

As used herein, the term “sample” and “specimen” are used interchangeably, and in the broadest senses. In one sense, sample is meant to include a specimen or culture obtained from any source, as well as biological and environmental samples. Biological samples may be obtained from animals (including humans) and encompass fluids, solids, tissues, and gases. Biological samples include blood products, such as plasma, serum, stool, urine, and the like. Environmental samples include environmental material such as surface matter, soil, mud, sludge, biofilms, water, and industrial samples. Such examples are not however to be construed as limiting the sample types applicable to the present invention.

The term “system” as used herein refers to a collection of compositions, devices, articles, materials, etc. grouped together in any suitable manner (e.g., physically associated; in fluid-, electronic-, or data-communication; packaged together; etc.) for a particular purpose.

### DETAILED DESCRIPTION

Provided herein are methods for detecting a target RNA in a sample. The methods described herein enable rapid transcription and polymerase chain reaction with a single enzyme, rather than one enzyme for reverse transcription and one enzyme for polymerase chain reaction. The methods comprise providing a reaction mixture containing the sample, amplification reagents, and a polymerase enzyme having both RNA and DNA-dependent polymerase activity; reverse transcribing the RNA to DNA by incubating for a reverse transcription time (e.g., for no longer than 5 minutes); and amplifying the DNA by performing a thermal cycling protocol comprising a plurality of amplification cycles, wherein each amplification cycle comprises at least a denaturation step and an annealing step.

a. Reaction Mixture

Any suitable PCR reagent may be used in the reaction mixture. Suitable PCR reagents include water, buffer, dNTPs, primers, controls, catalysts, initiators, promoters, cofactors, salts, chelating agents, probes, fluorescent dyes, and combinations thereof. For example, the reaction mixture may contain amplification reagents. The amplification reagents may include dNTPs, a buffer, a cofactor, and oligonucleotide primers configured for amplification of the target RNA in the sample.

The terms “primer” and “oligonucleotide primer” are used interchangeably herein. Generally, a primer is a shorter nucleic acid that is complementary to a longer template. During replication, the primer may be extended, based on the template sequence, to produce a longer nucleic acid that is a complementary copy of the template. Extension may occur by successive addition of individual nucleotides (e.g., by the action of a polymerase). A primer may be DNA, RNA, an analog thereof (e.g., an artificial nucleic acid), or any combination thereof. A primer may have any suitable length. For example, a primer may be at least 10 nucleotides. For example, a primer may be at least 10, at least 15, at least 20, at least 25, or at least 30 nucleotides. Exemplary primers are synthesized chemically.

Oligonucleotide primers may be supplied as at least one pair of primers for amplification of at least one nucleic acid target. For example, a pair of primers may be a forward primer (i.e. a sense primer) and a reverse primer (i.e. an antisense primer) that collectively define the opposing ends (and thus the length) of a resulting amplicon. Any suitable concentration of primers may be used. In some embodiments, the oligonucleotide primers are provided at a concentration of at least 6 μM. For example, the oligonucleotide primers may be provided at a concentration of at least 6 μM, at least 7 μM, at least 8 μM, at least 9 μM, at least 10 μM, at least 11 μM, or at least 12 μM. In some embodiments, the oligonucleotide primers are provided at a concentration of 12 μM.

The polymerase enzyme may be any suitable enzyme having both RNA and DNA-dependent polymerase activity. Polymerase enzymes having both DNA and RNA dependent polymerase activity may be commercially available polymerases (e.g., from Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). For example, the polymerase enzyme may be HawkZ05 Fast Polymerase. Any suitable concentration of polymerase enzyme may be used. In some embodiments, the polymerase enzyme may be provided at a concentration of at least 0.4 U/μL. For example, the polymerase enzyme may be provided at a concentration of at least 0.4 U/μL, at least 0.5 U/μL, at least 0.6 U/μL, at least 0.7 U/μL, or at least 0.8 U/μL. In some embodiments, the polymerase enzyme is provided at a concentration of 0.8 U/μL.

In some embodiments, the polymerase has both DNA and RNA dependent polymerase activity. In some embodiments, the polymerase is compatible with hot-start PCR. In some embodiments, the polymerase is part of an aptamer/enzyme system that allows for hot start PCR (e.g., polymerase is inactivated below a threshold temperature). In some embodiments, a polymerase from Thermus species ZO5 is provided.

The cofactor may be any suitable cofactor for the polymerase enzyme used. For example, the cofactor may be a magnesium salt. For example, the magnesium salt may be MgCl₂ or MgSO₄. As another example, the cofactor may be a manganese salt. For example, the manganese salt may be MnCl₂ or MnSO₄. Any suitable concentration of cofactor may be used. In some embodiments, the cofactor is provided at a concentration of 3 mM to 8 mM. For example, the cofactor may be provided at a concentration of 3 mM, 4 mM, 5 mM, 6 mM, 7 mM, or 8 mM. In some embodiments, the cofactor is provided at a concentration of 4 mM.

In accordance with the embodiments provided herein, PCR reagents can also include one or more probes, or any nucleic acid connected to at least one label, such as at least one dye. A probe may be a sequence-specific binding partner for a nucleic acid target and/or amplicon. The probe may be designed to enable detection of target amplification based on fluorescence resonance energy transfer (FRET), including one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe). The nucleic acid portion of the probe may have any suitable structure or origin, for example, the portion may be a locked nucleic acid, a member of a universal probe library, or the like. In other cases, a probe and one of the primers of a primer pair may be combined in the same molecule. For example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)).

b. Reverse Transcription

Reverse transcription refers to the process of generating a complementary DNA strand (cDNA) from the RNA template present in the sample. The methods described herein require a short incubation time to generate a cDNA product from the RNA template. In particular, the disclosed methods comprise reverse transcribing the RNA to DNA by incubating for a reverse transcription time (e.g., for no longer than 5 minutes). For example, the reverse transcription time may be no longer than 5 minutes, no longer than 4 minutes, no longer than 3 minutes, no longer than 2 minutes, no longer than 90 seconds, no longer than 60 seconds, no longer than 30 seconds, no longer than 15 seconds, no longer than 12 seconds, no longer than 10 seconds, no longer than 8 seconds, no longer than 5 seconds, or no longer than 1 second. In some embodiments, the reverse transcription time 0 seconds.

The reverse transcription step can occur at any suitable temperature dependent on the polymerase enzyme used. In some embodiments, the reverse transcription step is performed at an elevated temperature compared to the temperature typically used for methods of reverse transcription. For example, the HawkZ05 Fast Polymerase is sold with aptamer that prevents enzymatic activity below 55° C. Accordingly, for methods using the HawkZ05 Fast polymerase the reverse transcription step is performed at a temperature above 55° C. In some embodiments, the reverse transcription step may be performed at a temperature of 60-70° C. For example, the reverse transcription step may occur at a temperature above 55° C., above 60 ° C., or above 65° C. In some embodiments, the reverse transcription step occurs at a temperature of 68° C. Other polymerases may require alternative temperatures for the reverse transcription step.

Certain RNA targets require antecedent denaturation of the RNA prior to adding the RNA to the RT-PCR reaction. For example, denaturation of rotavirus or the RNA secondary structure seen in hepatitis C virus requires melting temperatures significantly above the optimal temperature range of commonly used reverse transcription enzymes. This leads to denaturation of the reverse transcription polymerase enzyme. For example, commonly used reverse transcription enzymes such as Maloney murine leukemia virus (MMLV) reverse transcriptase or avian myeloblastoma virus (AMV) reverse transcriptase have an optimal temperature range of 37-42° C. Accordingly, targets such as rotavirus or hepatitis C require a preliminary RNA denaturation step, a process which is not compatible for a one step, closed cartridge design. In contrast, the methods described herein enable the reverse transcription step to be performed at an elevated temperature range, such as in the range of 60-70° C. This allows for reverse transcription and subsequent detection of RNA targets without the need for antecedent denaturation of the RNA.

c. DNA Amplification

PCR reactions also generally involve a process of amplification, or a reaction in which replication occurs repeatedly over time to form multiple copies of at least one segment of a template molecule. Generally, amplification relies on alternating cycles of heating and cooling (i.e., thermal cycling) to achieve successive rounds of replication.

The methods disclosed herein comprise amplifying the DNA by performing a thermal cycling protocol comprising a plurality of amplification cycles. The amplification may be performed using any suitable reagents as described above. Each amplification cycle comprises at least a denaturation step and an annealing step. For example, each amplification cycle may alternate between two or more temperature set points, such as a higher melting (denaturation) temperature and a lower annealing/extension temperature. In other embodiments, each amplification cycle may alternate among three or more temperature set points, such as a higher melting temperature, a lower annealing temperature, and an intermediate extension temperature.

Any appropriate temperature and duration for each step in the amplification cycle may be used. Generally, each denaturation step is performed at a temperature from 91-98° C. For example, each denaturation step may be performed at 91° C., 92° C., 93° C., 94° C., 95° C., 96° C., 97° C., or 98° C. In some embodiments, each denaturation step is performed at 95° C. In some embodiments, each denaturation step may be performed for less than 20 seconds. For example, each denaturation step may be performed for less than 20 seconds, less than 10 seconds, less than 5 seconds, less than 4 seconds, less than 3 seconds, less than 2 seconds, or 1 second. In some embodiments, each denaturation step is performed for 1 second at 95° C.

The appropriate annealing temperature is dependent on the primer pair and may generally be performed at 45-70° C. For example, each annealing step may be performed at a temperature of 45° C., 50° C., 55° C., 58° C., 60° C., 65° C., or 68° C. Each annealing step may be performed for less than 20 seconds. For example, each annealing step may be performed for less than 20 seconds, less than 10 seconds, or less than 5 seconds. In some embodiments, each annealing step is performed for 4 seconds at 68° C.

In some embodiments, the thermal cycling protocol may comprise an initial hold at a high temperature (e.g. 95° C.) prior to performing the plurality of amplification cycles. For example, the thermal cycling protocol may comprise an initial hold at 95° C. for 2 minutes or less. In some embodiments, the thermal cycling protocol may comprise an initial hold for 2 minutes, 90 seconds, 1 minute, 30 seconds, or 15 seconds at 95° C.

Amplification may generate an exponential or linear increase in the number of copies as amplification proceeds. Typical amplifications produce a greater than 1,000-fold increase in copy number and/or signal. Any suitable number of amplification cycles may be performed to generate the desired signal. For example, the thermal cycling protocol may comprise at least 30 amplification cycles. In some embodiments, the thermal cycling protocol comprises 40 amplification cycles.

d. Devices

The disclosed methods may be performed using any suitable device. For example, a suitable device for performing the disclosed RT-qPCR methods may comprise a sample container, a first temperature zone, a second temperature zone, and a shuttling mechanism. The shuttling mechanism physically moves the sample container between the first and second temperature zones. The sample container may be a well capable of containing a liquid sample. Alternatively, the sample container may be a porous material capable of adsorbing a liquid sample. Each temperature zone may contain a temperature regulator that maintains a fixed temperature within a temperature zone. In some embodiments, suitable devices further comprise a detection zone, such as a detection zone comprising a fluorometer. In some embodiments, one or both of the temperature zones may be a detection zone. Exemplary devices are described in International Application No. PCT/US2018/034443, the entire contents of which are incorporated herein by reference.

e. Kits

The PCR reagents described herein may be incorporated into a kit for rapid detection of a target RNA in a sample. For example, the disclosed components may be incorporated into a kit for rapid clinical diagnostics, such as for detection of viral RNA in sample. Suitable kits may contain any appropriate primers and/or probes for detection of any desired RNA in the sample. For example, the kit may contain the appropriate components for detection of viral RNA that requires elevated temperatures (e.g. 60-70° C.) for denaturation of the RNA. Such kits would enable rapid reverse transcription and amplification of RNA targets such as rotavirus or hepatitis C virus without the need for an antecedent RNA denaturation step. In some embodiments, the kit may comprise the appropriate PCR reagents in a single closed cartridge for the detection of target RNA in a sample.

### EXAMPLES

### Example 1

### Fast RT-qPCR Assay

### Methods

HawkZ05 Fast Polymerase is marketed as a fast RT-qPCR assay. The manufacturer recommends performing the RT step for 2 to 5 minutes (4). It was tested how reaction conditions involving higher levels of primer and enzyme would affect the RT-qPCR assay. Surprisingly, very similar Cqs were measured when the RT time was 5 min, 2 min, 30 sec., 12, sec. and 5 sec. MS2 RT-qPCR primers and probes adapted from Beck, et al. (5) were used to amplify RNA extracted from MS2 bacteriophage. PCR data analyzed using LinRegPCR (6, 7). A prototype instrument was used to perform RT-qPCR with 15 μl samples. The RT step was performed at 68° C. from 0 seconds to 5 minutes, a 15 second hold at 95° C. and then 40 cycles of 1 second at 95° C. and 4 seconds at 68° C.

### Reaction Composition

The RT-qPCR Reaction Composition included the Following:
10% glycerol (ACROS)
0.2% Tween 20 (Pierce)
150 mM Trehalose (Life Sciences Advanced Technologies)
6 mg/ml ultrapure BSA (Ambion)
65 mM Tris pH 8.0 (Thermo Scientific)
62.4 mM Bicine/KOH pH 8.0 (USB)
65 mM potassium glutamate (Sigma)
0.4 mM dNTPs (Thermo Scientific)
4.0 mM MnCl₂ (Sigma)
12 μM MS2 forward and reverse primer mix (IDT; see sequence below)
500 nM MS2 FAM-labeled hydrolysis probe (IDT; see sequence below)
0.8 U/μL HawkZ05 Fast Polymerase (Roche Custom Biotech)
˜5000 copies MS2 RNA isolated from MS2 bacteriophage (Zeptometrix 0810066) by Dynal MyOne Silane Viral Isolation kit (Thermo Scientific)

Forward primer:
5′-agg tcg gta cta aca tca agt-3′
Reverse primer:
5′-gat atg ttg cac gtt gtc tgg a-3′
Hydrolysis probe:
5′-/56-FAM/cgt ctg tcg/zen/tat cca
gct gca aac t/3IABkFQ-3′

### Results

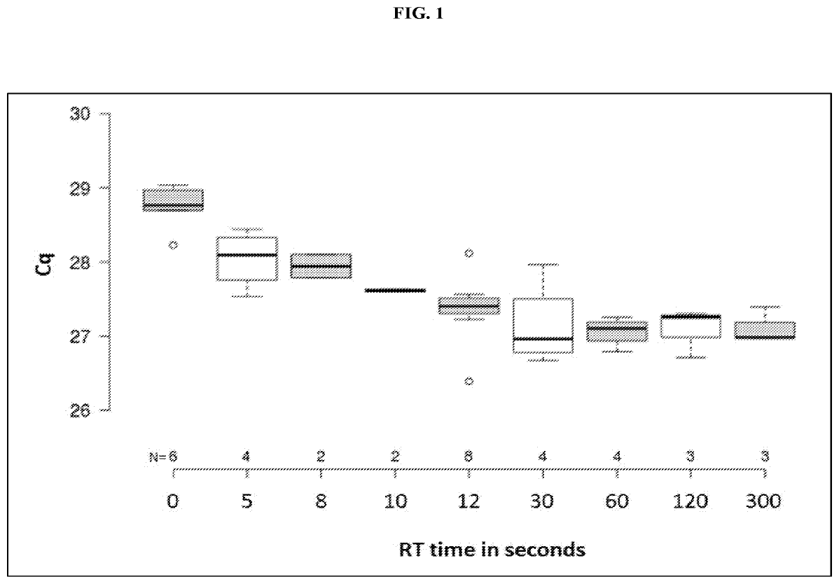

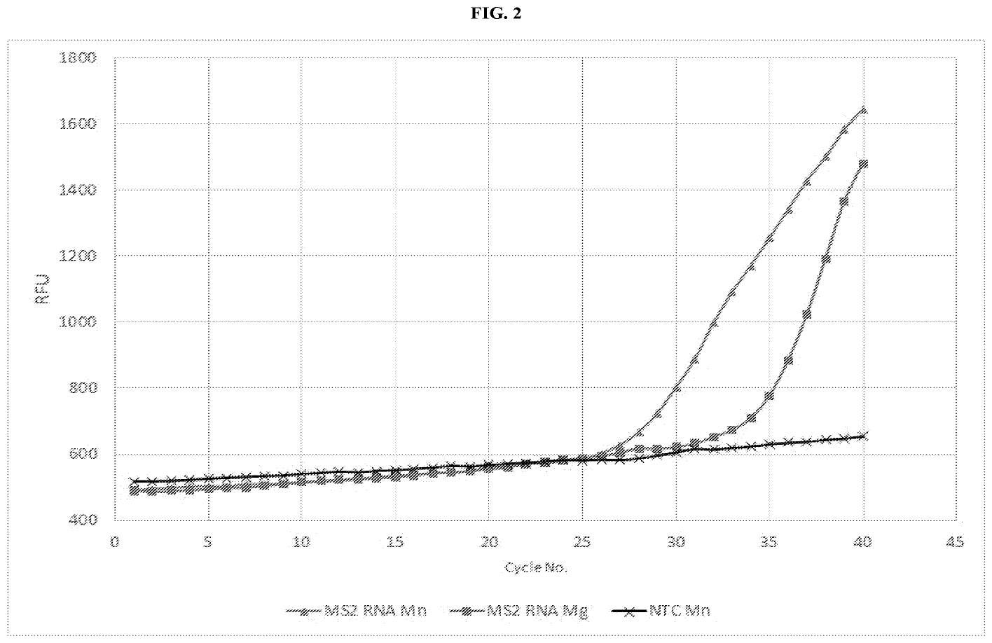

There was no difference in the Cq measured of the RT-qPCR assays of RT length from 5 seconds to 5 minutes (300 seconds) (FIG. 1). Substantial RT activity can be observed even with a 0 second hold at 68C. However, the difference between the average 12 second RT and 0 second RT was 1.4 Cq which corresponds to approximately 2.5-3-fold less yield of cDNA.
Surprisingly, a 0 second RT paired with Fast PCR cycling conditions yielded a product. ZO5′s cofactor of choice is Mg²⁺ for PCR and Mn²⁺ for RT-PCR. Therefore, to test if contaminating phage DNA were responsible for the Cq observed with 0 seconds RT, the MS2 RNA was amplified using the Mg²⁺ cofactor and compared the results to the Mn²⁺ cofactor. The PCR curve of the Mn²⁺ cofactor reaction rose up out of the background ˜6 cycles before the Mg²⁺ cofactor reaction FIG. 2). This difference in amplification corresponds to ˜2 orders of magnitude difference in amount of cDNA produced during RT step. The lack of amplification in the No Template Control reaction (NTC) demonstrates that the reagents were not contaminated with MS2 DNA.

A similar study was performed using Hepatitis C in vitro transcribed RNA that has been treated with DNase I as part of the transcription protocol (MEGAshortscript kit, Applied Biosystems). The DNA Mn²⁺ is a positive control to demonstrate that Mn²+ cofactor is acceptable in the PCR assay. The other two curves show that the RNA amplified with Mn²⁺ as the cofactor has much earlier visible amplification than the reaction with Mg²⁺ as cofactor, and the estimated Cqs would be 8-10 apart which is a fraction of a percent of the yield of RNA. with Mn²⁺ as the cofactor.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

### REFERENCES

The following references, some of which are cited above, are herein incorporated by reference in their entireties.
1. Farrar J S, Wittwer C T. Extreme PCR: efficient and specific DNA amplification in 15-60 seconds. Clinical chemistry. 2015;61(1):145-53.
2. Wittwer C T, Houskeeper J A, Myers Bigel P A, inventors; University of Utah Research Foundation, assignee. Extreme Reverse Transcription PCR2017 11 May 2017.
3. Mijatovic-Rustempasic S, Tam K I, Kerin T K, Lewis J M, Gautam R, Quaye O, et al. Sensitive and specific quantitative detection of rotavirus A by one-step real-time reverse transcription-PCR assay without antecedent double-stranded-RNA denaturation. Journal of clinical microbiology. 2013;51(9):3047-54.
4. Nakerakanti S, Sammeta N. Guidelines for the optimization of assays using HawkZ05 Fast Polymerase. In: Biotech RC, editor. 2018.
5. Beck E T, Jurgens L A, Kehl S C, Bose M E, Patitucci T, LaGue E, et al. Development of a rapid automated influenza A, influenza B, and respiratory syncytial virus A/B multiplex real-time RT-PCR assay and its use during the 2009 H1N1 swine-origin influenza virus epidemic in Milwaukee, Wisconsin. The Journal of molecular diagnostics: JMD. 2010;12(1):74-81.
6. Ramakers C, Ruijter J M, Deprez R H, Moorman A F. Assumption-free analysis of quantitative real-time polymerase chain reaction (PCR) data. Neuroscience letters. 2003;339(1):62-6.
7. Ruijter J M, Ramakers C, Hoogaars W M, Karlen Y, Bakker O., van den Hoff M J, et al. Amplification efficiency: linking baseline and bias in the analysis of quantitative PCR data. Nucleic acids research. 2009;37(6):e45.

1. A method for detecting a target RNA in a sample comprising:
a. Providing a reaction mixture containing the sample, amplification reagents, and a polymerase enzyme having both RNA and DNA-dependent polymerase activity;
b. Reverse transcribing the RNA to DNA by incubating for a reverse transcription time of no longer than 5 minutes;
c. Amplifying the DNA by performing a thermal cycling protocol comprising a plurality of amplification cycles, wherein each amplification cycle comprises at least a denaturation step and an annealing step.

2. The method of claim 1, wherein the amplification reagents comprise deoxynucleotide triphophates, a buffer, a cofactor, and oligonucleotide primers configured for amplification of the target RNA in the sample.

3. The method of claim 2, wherein the oligonucleotide primers comprise a forward primer and a reverse primer.

4. The method of claim 2, wherein the oligonucleotide primers are provided at a concentration of at least 6 μM.

5. The method of any one of claims 1-4, wherein the oligonucleotide primers are provided at a concentration of 12 μM.

6. The method of any one of claims 1-5, wherein the polymerase enzyme is provided at a concentration of at least 0.4 U/μL.

7. The method of any one of claims 1-6, wherein the polymerase enzyme is provided at a concentration of 0.8 U/μL.

8. The method of any one of claims 1-7, wherein the cofactor is a magnesium salt or a manganese salt.

9. The method of claim 8, wherein the cofactor is a manganese salt.

10. The method of claim 9, wherein the manganese salt is MnCl₂.

11. The method of any one of claims 1-10, wherein the cofactor is provided at a concentration of 3 mM to 8 mM.

12. The method of claim 11, wherein the cofactor is provided at a concentration of 4 mM.

13. The method of any one of claims 1-12, wherein the reverse transcription time is no longer than 2 minutes.

14. The method of claim 13, wherein the reverse transcription time is no longer than 30 seconds.

15. The method of claim 14, wherein the reverse transcription time is no longer than 12 seconds.

16. The method of claim 15, wherein the reverse transcription time is no longer than 5 seconds.

17. The method of claim 16, wherein the reverse transcription time is no longer than 1 second.

18. The method of any one of claims 1-17, wherein the reverse transcribing step occurs at a temperature of 68° C.

19. The method of any one of claims 1-18, wherein each denaturation step is performed for 1 second at 95° C. and each annealing step is performed for 4 seconds at 68° C.

20. The method of any one of claims 1-19, wherein the thermal cycling protocol comprises at least 30 amplification cycles.

21. The method of claim 20, wherein the thermal cycling protocol comprises 40 amplification cycles.
